(19) United States
(12) Patent Application Publication
(10) Pub. No.: US 2017/0164719 A1
(43) Pub. Date: Jun. 15, 2017

(54) Telescoping Personal Hygiene Mirror

(71) Applicant: Down Under Wonder, LLC, Snowmass, CO (US)

(72) Inventor: Patti C. Wheeler, Snowmass, CO (US)

(21) Appl. No.: 15/381,056

(22) Filed: Dec. 15, 2016

Related U.S. Application Data
(60) Provisional application No. 62/267,356, filed on Dec. 15, 2015.

(51) Int. Cl. A45D 42/10; A61B 6/06; F21V 33/00; H04N 5/225; G02B 7/182; F21V 8/00; G02B 5/08; A45D 42/16; F21V 23/04

(52) U.S. Cl. CPC A45D 42/10; A45D 42/16; A61B 6/06; F21V 33/004; F21V 23/0485; G02B 7/182; G02B 6/001; G02B 5/0808; H04N 5/2252; H04N 5/23245

(57) ABSTRACT

A personal hygiene device includes a head with a mirror affixed to a base by a telescopic arm, which is collapsible into the base for storage. The device includes at least one light and the mirror includes a capacitive region for touch-sensitive operation of the light. The device is waterproof for use in a bathroom. The device is optionally enabled for short range wireless communication with external devices, such as a smart phone with a related mobile device application. Optical features include a camera for capturing still, video, or panoramic images, which may be transmitted using the application, and a loudspeaker for playing sound via the application.

### CROSS REFERENCE TO RELATED APPLICATIONS

The present application claims priority to U.S. Provisional Patent Application No. 62/267,356 filed 15 Dec. 2015, which is hereby incorporated herein by reference in its entirety.

### TECHNICAL FIELD

The technology described herein relates to telescoping mirrors and methods of using the same.

### BACKGROUND

Some medical and personal hygiene procedures require an individual to view difficult-to-see anatomical regions. A handheld mirror may aid in visualization, but some of these procedures, such as examination, palpation, self-catheterization, or shaving, are easier to perform if both of a user's hands are free. The procedures may be performed in a medical setting or in a personal bathroom, including a shower, where direct lighting of an area of interest is not available. Plug-in lighting is unsafe in a wet bathroom.

An individual may be uncomfortable or embarrassed performing some examination procedures in front of another person. Indirect visualization, such as with photographs or video recordings, allows an individual to share images without the discomfort of being physically proximate to another person.

The information included in this Background section of the specification, including any references cited herein and any description or discussion thereof, is included for technical reference purposes only and is not to be regarded as subject matter by which the scope of the invention as defined in the claims is to be bound.

### SUMMARY

The technology disclosed herein relates to personal hygiene devices. The personal hygiene devices may be used to assist visualization of difficult-to-see anatomical regions while leaving the user's hands free to perform a personal care procedure. While the discussion herein is focused on the effectiveness of the devices when personal hygiene procedures are performed, it should be understood that the devices may also be used when medical procedures are performed.

In some embodiments, the personal hygiene device comprises a head, a base, and an arm connecting the head and the base. The head includes a mirror and at least one light. The mirror permits visualization of otherwise difficult-to-see areas of the body. The light illuminates at least the region being reflected in the mirror. The arm telescopes and can be collapsed and stored in the base when the device is closed. When the device is open, the arm can be extended. The arm is pivotable relative to the base and rotatable relative to the head.

In some embodiments, the device includes a rechargeable battery and a charging port for supplying power to the battery. In these embodiments, the device is portable and the light can be used when the device is not plugged in.

In some embodiments, the device is portable and includes an alkaline battery for supplying power to the device.

In some embodiments, the mirror includes a capacitive region for touch-sensitive operation of a light.

In some implementations, the personal hygiene device is water-resistant or waterproof.

In some embodiments, the device includes a speaker for playing instructions, music, or other sounds.

In some embodiments, the device includes a camera for capturing still, video, or virtual reality (360°) images. The device may also include media storage for storing the images from the camera.

In some embodiments, the personal hygiene device includes a short range transceiver (e.g. Bluetooth-enabled for communication with an external device, such as with a smart phone via a mobile device application). Images captured by the camera may be sent to an external device through the application. The application may control the function of the camera. Sounds from an external device may be sent to the personal hygiene device and played on the speaker through the application.

In one implementation, a method for using the personal hygiene device, such as to visualize an anatomical area of interest, is provided. The device, in the closed position, is placed in a desired location. The head is lifted upwards and away from the base, revealing the mirror and extending the arm. The head is directed to the desired position by pivoting the arm relative to the base and rotating the head omnidirectionally relative to the arm. The mirror reflects the anatomical area of interest and at least one light illuminates the area, which permits the user to visualize the area.

In another implementation, a method for using the personal hygiene device including a loudspeaker, such as to play music or other sounds, is provided. The head, base, and arm are used according to the method described above. Instructions, music, or other sounds are projected by the loudspeaker.

In another implementation, a method for using the personal hygiene device including a camera, such as to capture still, video, or panorama (virtual reality) images, is provided. The head, base, and arm are used according to any method described above. The camera captures still, video, or panorama (virtual reality) images and, in some embodiments, the images are also stored in a memory or other media storage device in the camera or in the personal hygiene device.

In another implementation, a method for using the personal hygiene device having short range wireless communication capabilities, such as to communicate with an external device is provided. The external device may be a smart phone having a mobile device application that interfaces with the personal hygiene device (e.g. a Bluetooth connection). The head, base, and arm are used according to any method described above. The loudspeaker is used according to the method described above and plays or broadcasts instructions, music, or other sounds via the application. The camera is used according to the method described above and its captured images can be visualized, transferred, or stored via the application.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

### BRIEF DESCRIPTION OF THE DRAWINGS

FIG. 1A is a top right isometric view of a personal hygiene device according to one embodiment in the collapsed position.

FIG. 1B is a top left isometric view of the personal hygiene device of FIG. 1A.

FIG. 2 is a bottom left isometric view of the personal hygiene device of FIG. 1A.

FIG. 3 is a left elevation view of the head positioning aid of FIG. 1A with the base removed.

FIG. 4 is a top left isometric view of the personal hygiene device of FIG. 1A with the head cover removed.

FIG. 5 is a bottom planar view of the head of the personal hygiene device of FIG. 1A with the mirror and head frame removed.

FIG. 6 is a cross-sectional view along line 6-6 in FIG. 1A.

FIG. 7 is a cross-sectional view along line 7-7 in FIG. 1B.

FIG. 8 is a cross-sectional view along line 8-8 in FIG. 1B.

FIG. 9 is a rear left view of the personal hygiene device of FIG. 1A in an open position.

FIG. 10 is a front left view of the of the personal hygiene device of FIG. 9.

### DETAILED DESCRIPTION

Personal hygiene devices primarily for use in visualizing difficult-to-see anatomical regions while performing medical or personal grooming activities are disclosed herein. Exemplary activities include dermatological examination, self-catheterization, Kegel exercises, sexual education, shaving, and waxing. The personal hygiene devices can be moved between an open position and a closed position. The devices are easy to transport and store and are also discreet when in the closed position. Visualization of difficult-to-see areas is permitted when the devices are in the open position.

### Components of the Personal Hygiene Device

The personal hygiene devices may be generally understood as having a head, a base, and an arm connecting the head and the base. The arm positions and holds the head in a desired position. The head provides a lid for the device when the device is in the closed position. The base provides a place to store the arm when the device is in the closed position and provides a counterweight to the head when the device is in the open position.

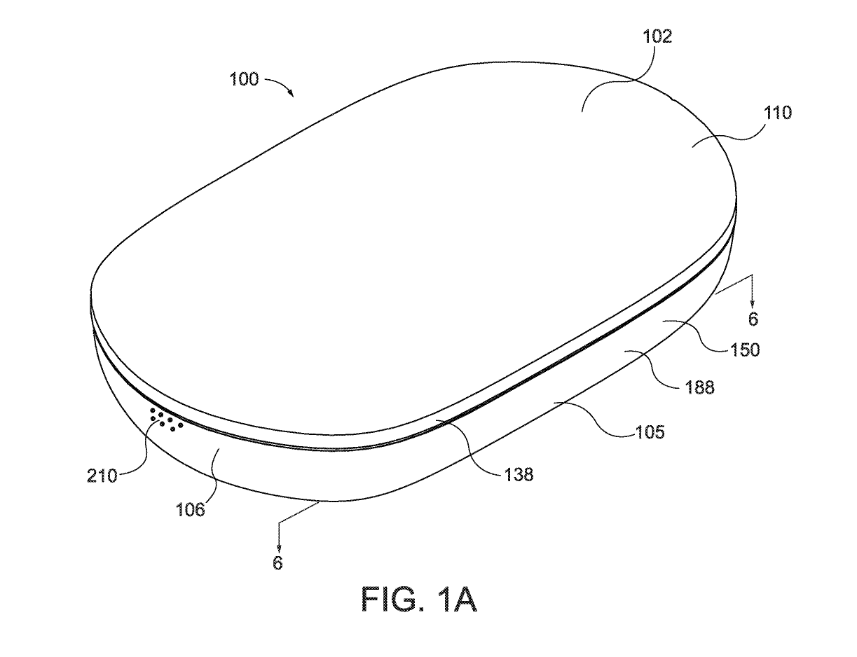

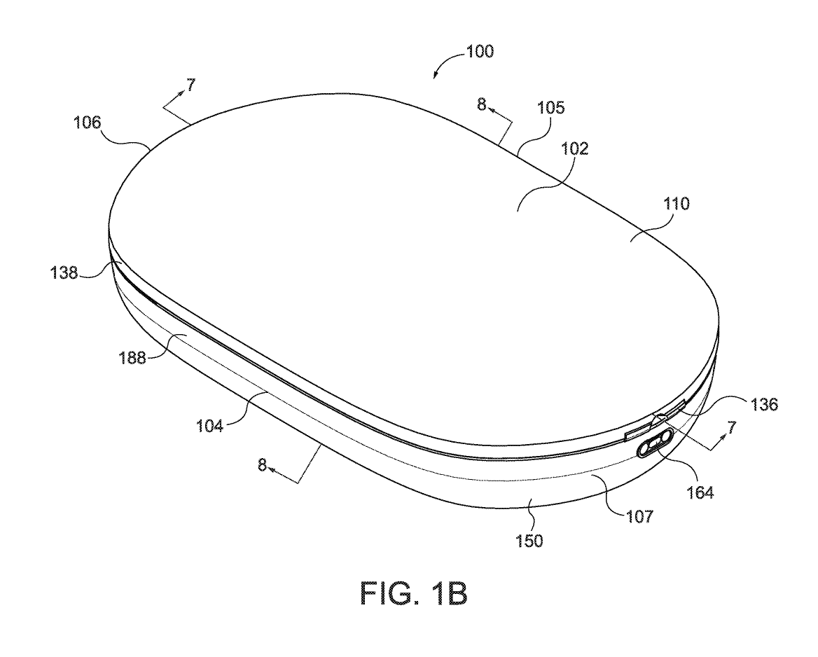

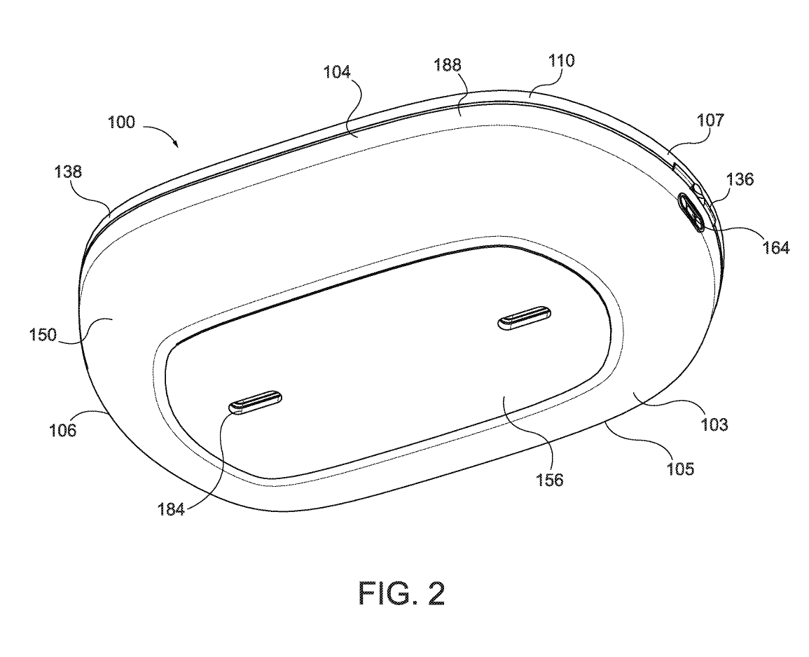

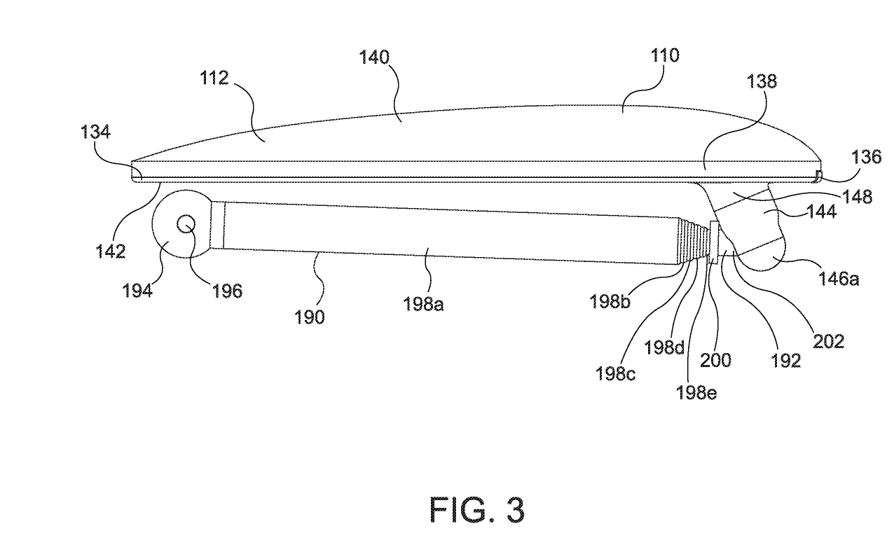

A personal hygiene device 100 according to one embodiment is shown in the closed position in FIGS. 1a, 1b and 2. When closed, the device 100 may have an overall ellipsoid shape with a smooth top side 102 that may be convexly curved or rounded such that its highest point is offset towards the back side 107 (see FIG. 3). The device 100 may also have a bottom side 103 that is flat in the center and curves gently outwards and upwards towards the top side 102 on each of the left side 104, right side 105, front side 106, and back side 107.

The device includes a head 110, a base 150, and an arm 190. When the device 100 is assembled and in the closed position, the head 110 may be positioned on top of the base 150. The height (measured along a vertical axis of the device 100 between the bottom side 103 and the top side 102) of the head 110 may account for about half or less than half the overall height of the device 100. The base 150 may account for about half or more than half the overall height of the device 100. The shape of a head perimeter 138 may be substantially the same as the shape of a base perimeter 188 such that the head perimeter 138 aligns with the base perimeter 188 when the device 100 is in the closed position. The head 110 may be flush with the base 150 along some or all of the left side 104, right side 105, front side 106, and back side 107. The alignment of the head 110 with the base 150 may help create a smooth and sleek external appearance for the device 100.

With reference to FIG. 3, the head 110 may include an upper surface 140 and a lower surface 142. The upper surface 140 may be coextensive with the top side 102 of the device 100.

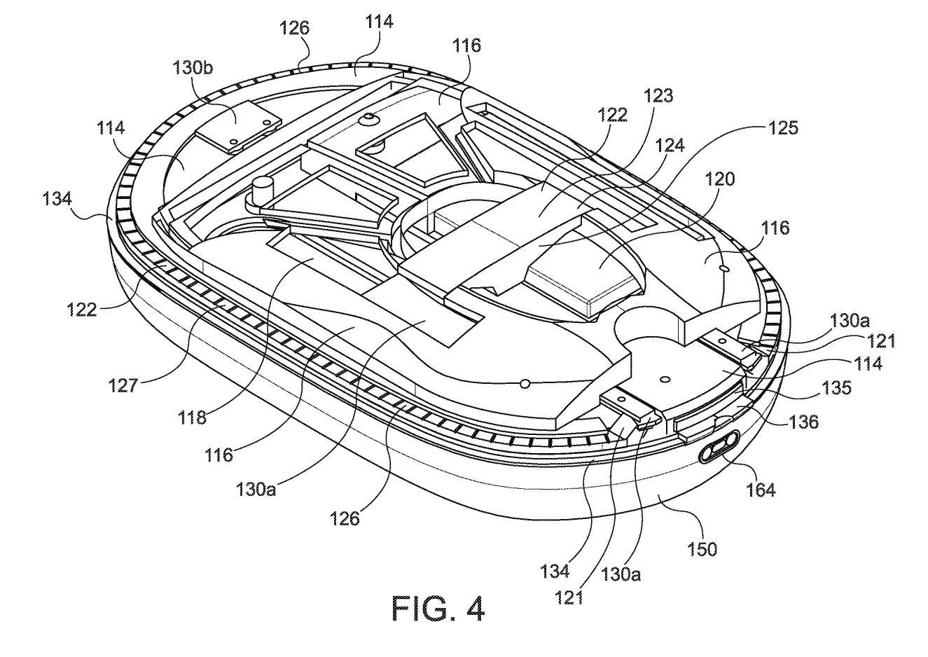

With reference to FIGS. 4, 5, 7, and 8, the head 110 may include a head frame 114 and a head subframe 116. Each of the head frame 114 and head subframe 116 may provide structural support or rigidity to the head 110. Also, each of the head frame 114 and head subframe 116 may provide a surface for mounting or otherwise securing additional components of the head 110, such as a head cover 112, a mirror 134, a head energy generator 118, a power source 120, one or more illumination systems 122, and one or more head control boards 130. Each of the head frame 114 and head subframe 116 may include features that aid in providing support, rigidity, or component attachment points. Features may include walls, lips, ridges, spokes, and the like.

With reference to FIGS. 3, 7, and 8, the head 110 may include the head cover 112 and the mirror 134. The head cover 112 may help provide a smooth exterior surface for the device 100 and may protect other components of the head 110. The head cover 112 may have an oval shape when viewed from the top side 102 of the device 100. The cover 112 may be curved or rounded with a variable height such that its highest point is offset from center along a longitudinal axis of the head 110.

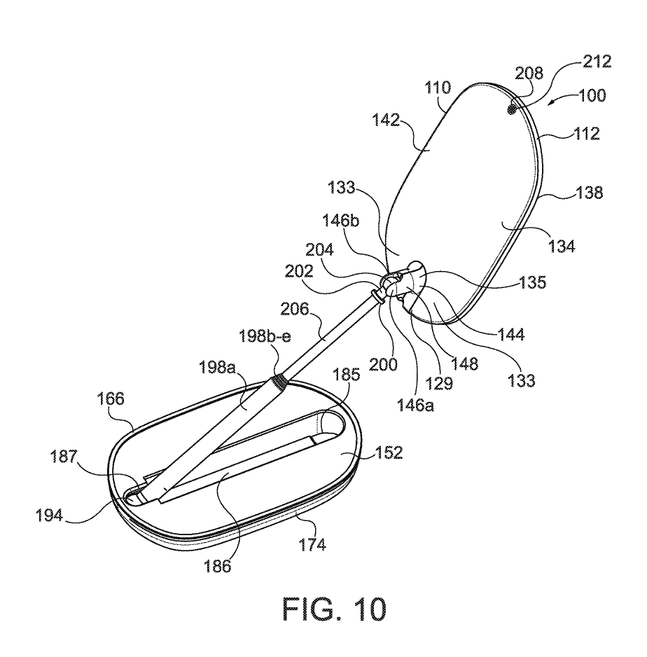

The mirror 134 may reflect and help a user visualize an anatomical area of interest. The mirror 134 may be substantially flat and oval in shape and may be constructed of a reflective material such as glass or plastic, which may be a polycarbonate. The material may also have fog-resistant properties. An arch-shaped cutout 129 may be formed in the mirror 134 as shown in FIG. 10.

The head 110 may include one or more illumination systems 122 as shown in FIGS. 4, 5, 7, and 8. Each illumination system 122 may include at least one light source 128 and at least one light distributor 127. Each light source 128 may provide light to the light distributor 127 to be used in illumination. Examples of a light source 128 include an LED, an incandescent bulb, a fluorescent bulb, or a compact fluorescent bulb. Each light distributor 127 may distribute light from the light source 128. Each light distributor 127 may be constructed of a transparent, reflective material, such as clear polycarbonate. The light distributor 127 may be designed for equidistribution of light along its length or for controlled light leakage. An example of a light distributor 127 is a light pipe.

In the embodiment depicted in FIGS. 1-10, the head 110 includes both a perimeter and a central illumination system 122. A perimeter light distributor 126 may be designed to illuminate at least the anatomical area reflected in the mirror 134 when the device 100 is in use. The perimeter light distributor 126 may have a broken oval ring shape with two ends 121. Two light sources 128a, 128b may provide light to the perimeter light distributor 126. A central light distributor 124 may be designed to provide a status indicator, such as of device 100 charging or a fully charged battery. The central light distributor 124 may have a substantially flat, rectangular crown 123 and an angled body 125 that conforms to the contours of adjacent components of the head 110. At least one light source 128f may provide light to the central light distributor 124.

The head 110 may also include a pin illumination system 212. A pin illumination system 212 may be designed to provide a small, focused amount of illumination on at least a portion of the anatomical area reflected in the mirror 134 when the device 100 is in use. A plurality of light sources 128c, 128d, 128e may provide light through a half-silvered (one-way) mirror (not shown) in the pin illumination system 212.

With reference to FIGS. 4, 5, 7, and 8, the head 110 may include a power assembly, which may include a head energy generator 118 and a power source 120. The power source 120 may be configured for receiving, storing, and supplying power. One example of a power source 120 is a rechargeable battery. Another example of a power source 120 is an alkaline battery or batteries, such as AAA or AA batteries. The head energy generator 118 may be configured for producing energy for storage in the power source 120. One example of a head energy generator 118 is an induction coil. The head energy generator 118 may be able to transmit energy, directly or indirectly, to the power source 120. The power source 120 may provide power to the device 100, such as for operating the one or more illumination systems 122. In the depicted embodiment, the head energy generator 118 has a flat oval shape and the power source 120 has a flat rectangular shape, but each may be any shape.

With reference to FIGS. 4, 5, 7, and 8, the head 110 may include one or more head control boards 130 for controlling the mechanical and electrical operation of the device 100. For example, a head control board 130b may control power to one or more light sources 128c, 128d, 128e in a pin illumination system 212. As another example, a head control board 130a may control touch-sensitive operation of one or more illumination systems 122. A head control board 130a may also sense when the head 110 is moved relative to the base 150 and may turn on or off an illumination system 122 in response. A head control board 130 may be a printed circuit board with a microcontroller or other processor (e.g., Reduced Instruction Set Microprocessor (RISC) or Application Specific Integrated Circuit (ASIC)) configured to manage such functionality.

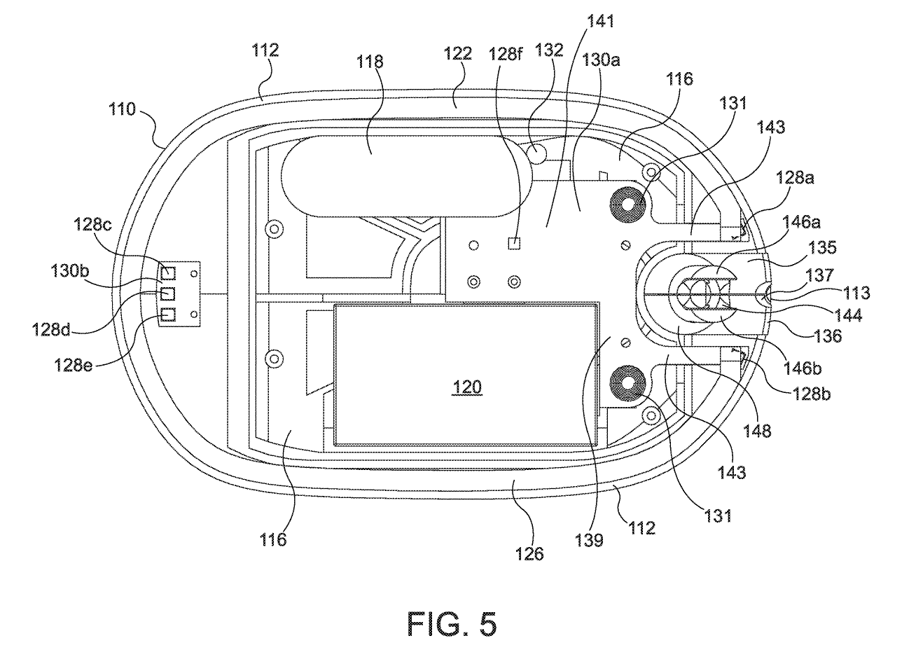

Related operational components, such as one or more light sources 128a, 128b or one or more touch sensors 131, may be mounted on a head control board 130. The touch sensors 131 may be responsive to touch, such as of a human hand or finger. In the design and construction of the head 110, the inclusion and placement of touch sensors 131 may create a capacitive region 133 that is responsive to human touch. With reference to FIGS. 5 and 10, two touch sensors 131 mounted on a head control board 130a may create one or more capacitive regions 133 on the lower surface 142 of the head, such as on the mirror 134.

As shown in FIG. 5, the head 110 may include a magnetic component 132. In conjunction with a complimentary magnetic material of or component in the base 150, the magnetic component 132 in the head 110 may help permit a secure close of the device 100.

With reference to FIGS. 3, 5, 7, and 10, the head 110 may include an arm connector 144, which may help secure the head 110 to the arm 190 and may help permit rotation of the head 110 about the arm 190. The arm connector 144 may include a body 148 from which two parallel legs 146a, 146b may extend. A hole 147, such as for receiving a screw or other component for helping to secure the arm connector 144 to the arm 190 or for providing tension to the arm 190, may be formed in the body 148. The body 148 may be topped by a cap 135 with a protruding lip 136 in which a notch 137 may be formed.

As shown in FIG. 10, the head 110 may include a camera 208 for capturing still, video, or panorama (virtual reality 360°) images. In some embodiments, the camera is configured to zoom, rotate, or pan to help produce a more precise photo or video. The device 100 may also include media storage (not shown) for storing the images from the camera 208. The camera 208 may be included instead of or in addition to the pin light illumination system 212.

Features and components of the base 150 will now be described. As shown in FIGS. 2 and 7-10, the base 150 may include a top surface 152 and a bottom surface 154. The bottom surface 154 of the base 150 may be coextensive with the bottom side 103 of the device 100.

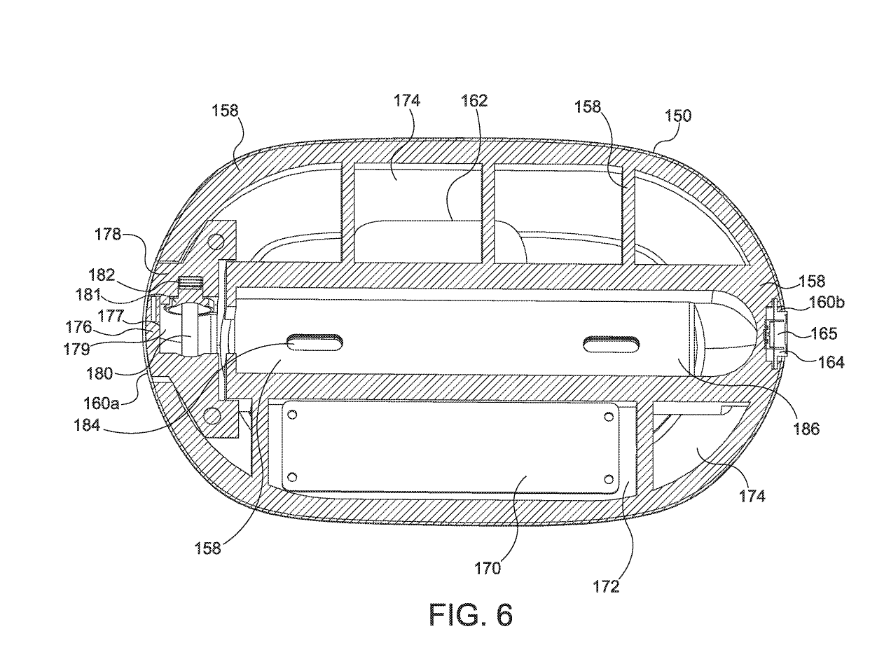

With reference to FIGS. 6-8, the base 150 may include a base frame 158, which may provide structural support or rigidity to the base 150. The base frame 158 may also provide a surface for mounting or otherwise securing additional components of the base 150, such as a base cover 174, a slip resistor 156, a mirror pad 166, a weight 162, a base energy generator 118, a base control board 170, a base control board cover 172, a socket assembly 176, and a port 164. The base frame 158 may include features that aid in providing support, rigidity, or component attachment points. Features may include walls, lips, ridges, spokes, and the like.

In the embodiment depicted in FIGS. 6-8, the base frame 158 has rounded sides at the left, right, front, and back sides 104, 105, 106, 107 of the device 100. A gap 160a, 160b may be formed in each of the front and back sides 104, 105.

The base frame 158 may be oval in shape when viewed from the top surface 152 of the base 150. A recessed rim 159 may extend around the base frame 158 near the top surface 152 and along the base perimeter 188. Interior to the recessed rim 159, the base frame 158 may be flat with a well 186 formed in it that extends from the top surface 152 towards the bottom surface 154 and from the front side 106 to the back side 107. The well 186 may be shaped like two arches placed base-to-base with a first arch 185 longer and wider than a second arch 187. The overall contours of the well 186, including variable length, width, and depth, may be complimentary to the shape of an arm 190. One or more drain holes 184 may be formed in the bottom of the base frame 158, such as in the first arch 185 of the well 186, for draining water that enters the base 150 from the top surface 152.

With reference to FIGS. 6-8, the base 150 may include a base cover 174. The base cover 174 may help provide a smooth exterior surface for the device 100 and may protect other components of the base 150. The base cover 174 may have an oval shape when viewed from the bottom side 103 of the device 100. The base cover 174 may be a formed as a shell with a bottom and sides but no top. One or more drain holes 184 may be formed in the bottom of the base cover 174 for draining water that enters the base 150 from the top surface 152.

At least a portion of the bottom surface 154 of the base 150 may comprise a slip resistor 156. The slip resistor 156 may help to prevent skidding or slipping of the device 100, especially when the device 100 is placed on a smooth or wet surface or is used in a wet environment. The slip resistor 156 may be formed as one or more pads integrated into the base cover 174 or may be a separate component secured to the base cover 174. In the embodiment depicted in FIGS. 2, 7, 8, and 10, the slip resistor 156 is seated in a recess in the base cover 174. The slip resistor 156 may be constructed of any material that provides traction or resists slippage, such as rubber. The remainder of the base cover 174 may be constructed of a rigid material such as hard plastic, stainless steel, or metal.

One or more suction cups (not shown) may be included on the bottom surface 154 to further help secure the device 100 when it is placed on a slanted, uneven, or slippery surface, such as a shower floor.

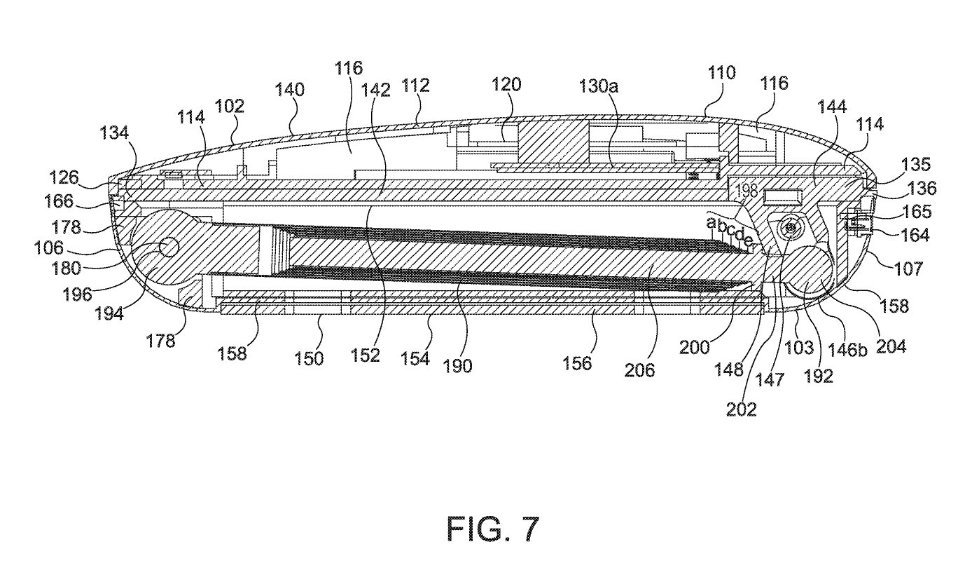

With reference to FIGS. 7, 8, and 10, the base 150 may include a mirror pad 166. The mirror pad 166 may be shaped as an oval ring. The mirror pad 166 may be constructed of any material that provides cushioning or impact absorption, such as rubber.

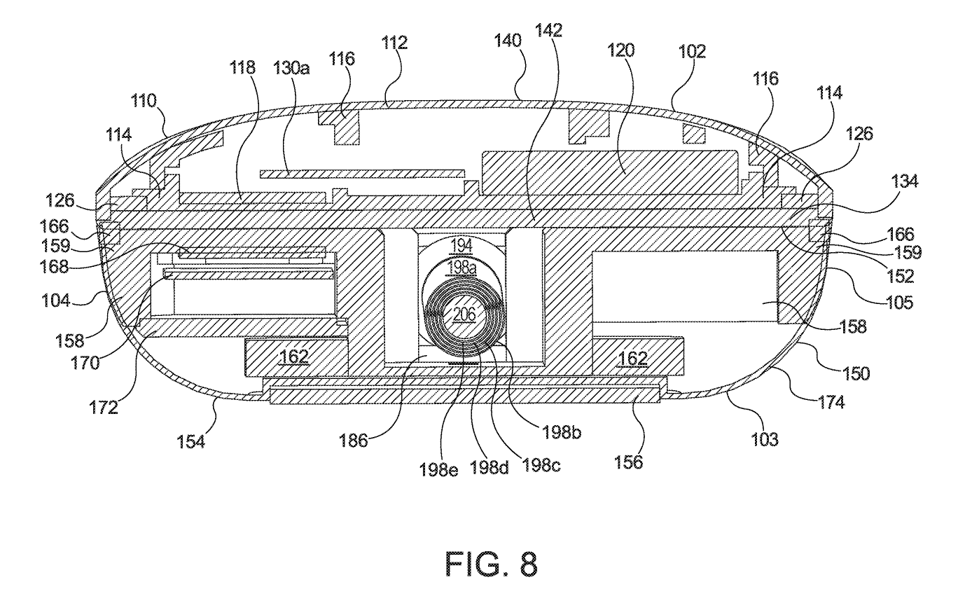

The base 150 may include one or more weights 162, as shown in FIGS. 6 and 8. Each weight 162 may help the base 150 remain in a desired location and not move or tip when the device 100 is in use. FIGS. 6 and 8 show two weights 162, each with a flat oval shape, but the weights 162 may be present in any number and shape.

With reference to FIG. 8, the base 150 may include a base energy generator 168, which may be configured for producing energy. One example of a base energy generator 168 is an induction coil. When connected to a power source (e.g. a wall outlet) the base energy generator 168 may be able to transmit energy, directly or indirectly, to the power source 120 in the head 110. In the depicted embodiment, the base energy generator 168 has a flat oval shape, but may be any shape. The type, shape, and size may be the same as that of the head energy generator 118.

With reference to FIGS. 6 and 8, the base 150 may include one or more base control boards 170 for controlling the mechanical and electrical operation of the device 100. For example, a base control board 170, or an electrical component thereof, may sense when a power source 120 is full, such as a fully charged battery, and may shut off a charging circuit. The base control board 170 may be a printed circuit board.

The base 150 may also include a base control board cover 172, which may help protect and seal the base control board 170 in a waterproof cavity.

With reference to FIGS. 6 and 7, the base 150 may include a socket assembly 176, which may secure the arm 190 to the base 150 and may permit pivoting of the arm 190 relative to the base 150. The socket assembly 176 may include an angular bracket 174 having inner walls 177 that define a cavity 179 through which a screw 180 passes. The screw 180 is secured to the angular bracket 178 and is encircled at one end by an annular ring 181 and one or more springs 182.

As shown in FIGS. 1B, 6, and 7, the base 150 may also include a port 164, such as for providing power to charge the device 100 and/or no transfer data to and from the device. A port 164 may be covered, for example, with a rubber plug, or otherwise closed to prevent water from entering the base 150. For example, the port 164 may be a magnetic USB port with no external openings and a female portion 165 of the USB internal to the base 150. The female portion 165 mates with a male portion on the charging cord (not shown). The USB may be a standard, mini, or micro USB or a lighting cable port or other type or protocol.

With reference to FIG. 1A, the base 150 may include a speaker 210 for playing instructions, music, or other sounds, such as from a synchronized mobile device application. The loudspeaker 210 may be sealed against the base 150 and have a waterproof cone or may be covered or otherwise closed to prevent water from entering the base 150. In the depicted embodiment, the loudspeaker 210 is sealed in or passes through the base cover 174.

Features and components of the arm 190 will now be described. The components of the arm 190 may be constructed of water-resistant or waterproof materials, such as plastic or stainless steel. As shown in FIGS. 3 and 7, the arm includes a cylindrical rod 206 with a base connector 194 at one end at a head connector 192 at the opposing end.

The base connector 194 may help secure the arm 190 to the base 150 and help the arm 190 pivot relative to the base 150. The base connector 194 may be substantially spherical in shape with an aperture 196 formed through it in a direction perpendicular to the length of the arm 190.

The head connector 192 may help secure the arm 190 to the head 110 and help the arm 190 and head pivot relative to each other. The head connector 192 may comprise a spherical ball 204, a neck 202, and an annular stop ring 200 at the base of the neck 202 where the neck 202 meets the rod 206.

The rod 206 may be encircled along its length by a plurality of concentric hollow tubes 198. Each tube 198 may be slightly shorter in length and wider in diameter than the immediately interior tube 198. For example, tube 198e may be positioned around and immediately exterior to rod 206. Tube 198d may be positioned around and immediately exterior to tube 198e. The diameter of tube 198d may be slightly larger than the diameter of tube 198e in order for tube 198e to fit inside of tube 198d. Tube 198d may be slightly shorter than tube 198e, which may be approximately as long as rod 206. Tube 198c may be positioned around and immediately exterior to tube 198d. The diameter of tube 198c may be slightly larger than the diameter of tube 198d and slightly smaller than the diameter of tube 198b. Tube 198c may be slightly shorter than tube 198d and slightly longer than tube 198b. Tube 198a may be the most exterior tube 198 as well as the shortest and widest. In the design and configuration of the rod 206 and tubes 198, an extendible and collapsible telescoping arm 190 may be formed. The arm 190 may extend from a length of approximately 5 to 7 inches to a length of approximately 10 to 30 inches.

When the device 100 is assembled, the components of FIGS. 1-10 may be assembled as follows.

With reference to FIGS. 1, 9, and 10, the head cover 112 may form a majority of the top side 102 of the device and the upper surface 140 of the head 110. The mirror 134 may form a majority of the lower surface 142 of the head 110. The mirror 134 may extend edge-to-edge across the lower surface 142 from the right side 105 of the device 100 to the left side 104. The mirror 134 may extend from the front side 106 to the back side 107 except for the cutout 129 positioned at the back side 107. The head cover 112 and the mirror 134 together may form a majority of the exterior of the head 110.

As shown in FIGS. 7 and 8, the head cover 112 may be secured to and supported by the head subframe 116, which may be positioned internal to and adjacent to the head cover 112. The mirror 134 may be secured to the head frame 114, which may be positioned internal to and adjacent to the mirror 134. The head subframe 116 may be positioned adjacent to the head frame 114 and closer to the upper surface 140 of the head 110 than the head frame 114 is positioned. The head frame 114 may be positioned closer to the lower surface 142 than the head subframe 116 is. The head cover 112, mirror 134, and other components of the head 110 may be secured to the head frame 114, head subframe 116, or each other by glue, screws, pins or dowels and complimentary holes, press fitting, and the like.

With reference to FIG. 4, the central light distributor 124 may be positioned between and adjacent to each of the head cover 112 and head subframe 116. The central light distributor 124 may positioned approximately in the center of the head 110 as measured from the left side 104 to the right side 105 of the device 100 and slightly offset towards the back side 107 as compared to the front side 106.

As shown in FIGS. 4, 5, 7, and 8, the perimeter light distributor 126 may be positioned along the head perimeter 138, adjacent to and internal to the mirror 134. The perimeter light distributor 126 may also be positioned adjacent to, exterior to, and in the same horizontal plane as the head frame 114. The ends 121 of the perimeter light distributor 126 may be proximate to the back side 107 of the device 100. A light source 128a, 128b, which may be mounted on a control board 130a, may be positioned at or near each end 121 of the perimeter light distributor 126. In the design and construction of the head 110 and the perimeter light distributor 126, the perimeter light distributor 126 may not be visible, through the mirror 134 or otherwise, unless the light sources 128a, 128b are turned on.

With continued reference to FIGS. 4, 5, 7, and 8, the head energy generator 118 may be seated in or otherwise secured to the head frame 114. A portion of the head energy generator 118 may be beneath and adjacent to a portion of head control board 130a. The head energy generator 118 may be positioned proximate to the left side 104 of the device 100 and closer to the front side 106 than the back side 107.

The power source 120 may be may be seated in or otherwise secured to the head frame 114. A portion of the power source 120 may be beneath and adjacent to a portion of the central light distributor 124. The power source 120 may be positioned proximate the right side 105 of the device 100 and closer to the back side 107 than the front side 106.

With reference to FIG. 5, the seat 139 and back 141 of the head control board 130a may border portions of two sides of the power source 120. A portion of the head control board 130a may be positioned beneath (toward the lower surface 142 of the head 110) the central light distributor 124 and another portion of the head control board 130a may be positioned above (toward the upper surface 140 of the head 110) a portion of the head energy generator 118. The legs 143 may extend toward or to the back side 107 of the device 100 and may flank the arm connector 144. A light source 128a, 128b may be secured on the underside of the terminal end of each leg 143. A touch sensor 131 may be secured to the underside of the seat 139 near the origin of each leg 143.

As shown in FIGS. 4 and 5, the head control board 130b may be secured to the head frame 114 and positioned towards the front side 106 of the device 100, approximately in the center between the left and right sides 104, 105.

With reference to FIG. 5, the magnetic component 132 may be secured to the head frame 114 and may be positioned towards the left side 104 of the device 100, adjacent the head energy generator 118 on the side of the head energy generator 118 facing the back side 107.

As shown in FIGS. 4, 5, and 7, the arm connector 144 may be positioned at the back side 107 of the device 100, approximately in the middle between the left and right sides 104, 105 and in the cutout 129 of the mirror 134. The body 148 and legs 146a, 146b may extend below the plane created by the lower surface 142 of the head 110. The cap 135 may be positioned above the lower surface 142 such that the lip 136 is received in a notch 113 in the head cover 112, which helps to secure the arm connector 144 and thereby the arm 190 to the head 110.

When the device 100 includes a camera 208, it may be positioned at the front side 106, approximately in the middle between the left and right sides 104, 105, and close to the lower surface 142 behind the mirror 134. A portion of the mirror backing may be removed to create a window through which the lens of the camera is exposed and can receive light for imaging.

With reference to FIGS. 6-8, the base frame 158, base cover 174, and slip resistor 156 together may form a majority of the exterior of the base 150. The base frame 158 may form a majority of the top surface 152 of the base 150. The base cover 174 and slip resistor 156 may form a majority of bottom side 103 of the device 100 and the bottom surface 154 of the base 150. The slip resistor 156 may be positioned on the bottom surface 154 midway between the front side 106 and back side 107, and midway between the left side 104 and right side 105.

With reference to FIGS. 7 and 8, the mirror pad 166 may sit in the recessed rim 159 of the base frame 158 and thereby extend around the base frame 158 near the top surface 152 and along the base perimeter 188.

As shown in FIG. 6, the drain holes 184 in the well 186 may align with the drain holes 184 in the base cover 174, which permits water that enters the well 186 to exit through the bottom surface 15 of the base 150.

As shown in FIGS. 6 and 8, each weight 162 may be positioned offset from center, slightly towards the front side 106, adjacent the base frame 158 on either side of the well 186.

With further reference to FIGS. 6 and 8, the base energy generator 168 may be positioned toward the right side 105 of the device 100, offset from center in the direction of the front side 106. The base control board 170 may be positioned directly beneath the base energy generator 168, closer to the bottom surface 154, and may extend beyond the base energy generator 168 in both width and length. The base control board cover 172 may be positioned adjacent the base frame 158, below the control board 170, and may extend beyond the control board 170 in both width and length.

As shown in FIGS. 6 and 7, the socket assembly 176 may be positioned at the front side 106 in the gap 160a in the base frame 158. The screw 180 runs perpendicular to the length of the well 186. The port 164 is positioned at the back side 107 in the gap 160b in the base cover 174.

As shown in FIG. 7, the arm 190, when collapsed, can be stored in the well 186 in the base 150. The contours of the well 186 may be complementary to the size and shape of the arm 190. The arm 190 is attached to the base 150 by an association or connection of the socket assembly 176 and the base connector 194. For example, the screw 180 may pass through the aperture 196 in the base connector 194 to help secure the arm 190 to the base 150. The annular ring 181 of the socket assembly 176 may be pushed up against the base connector 194 by the springs 182.

The arm 110 may be attached to the head 110 by an association or connection of the arm connector 144 and the head connector 192. For example, the ball 204 of the head connector 192 may be captured between the legs 146a, 146b of the arm connector 144.

When the device 100 is assembled, each of the head 110, base 150, and arm 190 may be water-resistant or waterproof. For example, the exterior of each of the head 110, base 150, and arm 190 may be constructed of a water-resistant or waterproof material. Seals between adjacent exterior components may exclude water and moisture from the interior of each of the head 110, base 150, and arm 190. Water is prevented from entering the charging port 164, camera 208, or loudspeaker 210. When the power source 120 is replaceable, such as alkaline batteries, water is prevented from contacting the power source 120. Water may be able to pass through the base 150 but only via the well 186 and the drain 184 holes in the bottom of well 186 and base cover 174.

When the device 100 is assembled and in the closed position, the lower surface 142 of the head 110 may be positioned above and adjacent to the top surface of the base 150, as shown in FIGS. 7 and 8. The mirror 134 may be adjacent to or in contact with the mirror pad 166, which may help protect the mirror 134 as it is being closed or when it is closed.

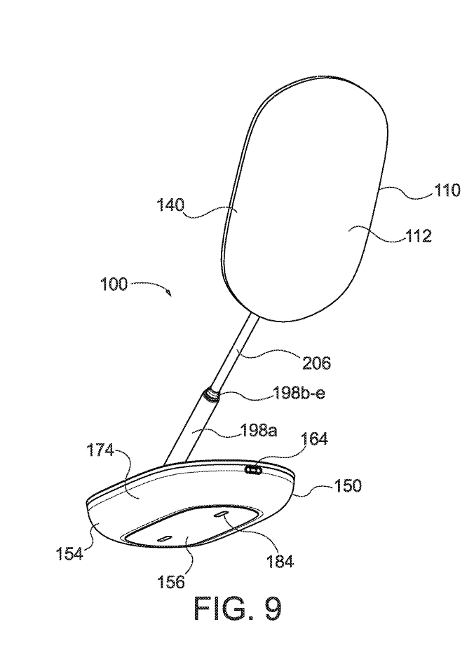

When the device 100 is assembled and in the open position, as show in FIGS. 9 and 10, the lower surface 142 of the head 110 is not positioned adjacent to the top surface of the base 150. Instead, the head 110 and base 150 are at least slightly physically separated and the orientation of the head 110 in relation to the base 150 may be changed compared to the orientation in the closed position. The arm 110 may permit the change in orientation of the head 110 relative to the base 150.

### Electrical Operation of the Personal Hygiene Device

By way of example, but not limitation, the personal hygiene device 100 of FIGS. 1-10 may be operated as follows. With the device 100 in the closed position, one end of a charging cord (not shown) may be plugged in to an electrical outlet and the other end may be applied to the port 164 and secured by magnetic attraction with the port 164. When the cord is secured to the port 164, electrical energy transfers from the cord to the port 164 to the base energy generator 168, through the top surface 152 of the base 150 and the lower surface 142 of the head 110 to the head energy generator 118, and then to the power source 120, where it charges the power source 120.

While the device is charging, light sources 128 may illuminate the central light distributor 142, such as continuously or in a pulsing pattern.

The base control board 170 may detect when the power source 120 is fully charged and may shut off the electrical circuit.

The device 100 may be operational while plugged in or unplugged, if the power source 120 is sufficiently charged. Charging the power source 120 and then unplugging the device 100 permits the device 100 to be transportable to any desired location for use.

Alternatively, the power source 120 may be an alkaline battery that does not require charging, and the device 100 does not need to be plugged in, prior to operation of the device 100.

When the device 100 is in the closed position (see FIGS. 1 and 2), head control board 130a may sense when the head 110 is then lifted away from the base 150, and may turn on the light sources 128a, 128b for the perimeter light distributor 126 in response. The head control board 130a may also sense when the lower surface 142, such as the mirror 134, of the head 110 closely approaches or comes in contact with the top surface 152 of the base 150 and may turn off the light sources 128a, 128b.

When the device is in the open position (see FIGS. 9 and 10), head control board 130a may control the response of the device 100 to touch, such as human touch. For example, head control board 130a may change the light mode in response to a finger applied with pressure to the mirror 134 in the capacitive region 133. Such pressure depresses one of the touch sensors 131 on the head control board 130a. For example, the control board 130a may direct the perimeter illumination system 122 off and the pin illumination system 212 on in response to a first application. The control board 130a may direct the perimeter illumination system 122 on in response to the second application. In response to the third application, both the perimeter illumination system 122 and the pin illumination system 212 may be turned off. One light mode may gradually transition to next selected mode.

### Methods of Use of the Personal Hygiene Device

By way of example, but not limitation, the personal hygiene device 100 of FIGS. 1-10 may be used to visualize an anatomical area of interest, such as while performing a personal grooming or medical task, according to the following procedure. The device 100 in the closed position (see FIGS. 1 and 2) may be placed in a desired location, such as the floor of a shower. The slip resistor 156 may stick to or grip the surface on which the base 150 is set, which helps maintain the device 100 in the desired location and prevent it from moving.

To move the device into the open position (see FIGS. 9 and 10), a user grasps the head 110, such as by the head cover 112 towards the front side 106. The base cover 174 may be notched (not shown) along the right side 105 and/or left side 104 to aid the user in grasping under the head cover 112.

The portion of the head 110 on the front side 106 of the device 100 is rotated upwards, approximately 90 degrees from a horizontal plane of base 150, before it is lifted upwards and away from the base 150. Rotating the head 110 reveals the mirror 134 on the lower surface 142 of the head 110. Alternatively, the head 110 is lifted relatively directly upwards from the base 150 without first pivoting the head 110.

Lifting the head 110 away from the base 150 automatically extends the arm 190. The arm 190 may be extended as much as desired by pulling on the head 110. The head 110 may then be directed to the desired position by pivoting the arm 190 up to 90 degrees relative to the base 150 and rotating the head 110 omnidirectionally relative to the arm 190. The desired location may be one in which an anatomical area of interest is reflected in the mirror 134.

When the head 110 is first dissociated form the base 150, the light sources 128a, 128b may automatically turn on and illuminate the perimeter light distributor 126.

The light mode may be changed by touching the mirror 134 in the capacitive region 133 as described above. The mirror 134 position may be adjusted such that the anatomical area of interest is illuminated by the perimeter light distributor 126 or the pin illumination system 212 or both 126, 212. A grooming task, such as shaving, or a medical task, such as self-catheterization, may be performed while the anatomical area of interest is reflected in the mirror 134 and visualized by the user.

The device 100 is returned to the closed position by pushing down on the head 110 and directing it towards the base 150, which causes the arm 190 to collapse. The head 110 is pushed such that the arm 190 collapses completely and is forced into the well 186 of the base 150. The mirror 134 in the head 110 is brought adjacent to the mirror pad 166 in the base 150. The mirror pad 166 protects the mirror 134 while the device 100 is being closed.

By way of example, but not limitation, the personal hygiene device 100 of FIGS. 1-10 may be used to play instructions, music, or other sounds, such as why visualizing an anatomical area of interest, according to the following procedure. The head 110, base 150, and arm 190 are used according to the procedure described above. A user may also play instructions, music, or other sounds through the loudspeaker 210 while using the device 100, for example, as transmitted by a short range wireless technology (e.g. Bluetooth).

In another example, the personal hygiene device 100 having Bluetooth capabilities, such as to communicate with an external device, is used according to the following procedure. The external device may be a smart phone having a mobile device application that interfaces with the personal hygiene device 100. The head 110, base 150, and arm 190 are used according to any procedure described above.

The loudspeaker 208 is used according to the procedure described above and plays instructions, music, or other sounds via the application. Instructions may be provided for performing activities such as Kegel exercises, self-catheterization, or sexual education.

By way of example, but not limitation, the personal hygiene device 100 of FIGS. 1-10 may be used to capture still, video, or virtual reality images, such as why visualizing an anatomical area of interest, according to the following procedure. The head 110, base 150, and arm 190 are used according to any procedure described above. The images are taken by the camera 208 and may be stored on media storage.

The camera 210 is used according to the method described above and its operation, such as focus, zoom, and shutter activation, may be controlled by the application on the mobile device. The captured images can be visualized, transferred, or stored via the application.

In another example, the personal hygiene device 100 having Bluetooth capabilities and a camera, such as to aid in providing medical services remotely, is used according to the following procedure. The head 110, base 150, arm 190, and camera 210 are used according to any procedure described above. The loudspeaker 208 may also be used as described above.

A medical professional, physically removed from the user of the device 100, views captured images, either stored or in real time, and provides feedback. For example, a dermatological region is visualized by the user of the device 100, the camera 210 captures images of the dermatological region and sends them to a remote medical professional (e.g., via a Bluetooth link with a smart phone and a related application), and the professional may provide a diagnosis of a skin condition. As another example, the urogenital region is visualized by the user of the device 100, the camera 210 captures images of the urogenital region and sends them to a remote medical professional, and the medical professional provides self-catheterization, Kegel exercise, sexual education, or other treatment or examination instructions, which are played through the loudspeaker 208, to the user.

### Kit Including the Personal Hygiene Device

In some embodiments, the personal hygiene device 100 is provided as part of a kit. The kit may also include a power cord for supplying electric power to the device 100, such as to charge the device 100. The kit may also include products, such as shaving cream or gel or waxing strips, that may be used while using the device 100 to perform a personal grooming task such as shaving or waxing. The kit may include a box in which the components are shipped or stored.

The article “a” or “an” preceding a term, as used herein, refers to one or more of that term. As such, the terms “a” or “an”, “one or more”, and “at least one” should be considered interchangeable herein.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A personal hygiene or examination device comprising
a head having a length and a width and further comprising
a mirror surface substantially coextensive with a first side of the head, wherein an first area of the mirrored surface is partially mirrored;
a power supply;
a touch-sensitive, capacitive region configured in an area of the mirror surface and connected to the power supply; and
at least one light source operatively controlled by the capacitive region;
a base defining a recessed well; and
a telescoping arm operatively connected between the head and the base; wherein
the base is configured to receive the arm in the recessed well when the arm is collapsed.

2. The personal hygiene device of claim 1 further comprising comprises a light pipe positioned behind the mirror to direct light from the light source to the partially mirrored area.

3. The personal hygiene device of claim 1, wherein the arm is affixed to each of the head and the base by a pivotable or rotatable connector.

4. The personal hygiene device of claim 1, wherein the device is water-resistant or waterproof.

5. The personal hygiene device of claim 1 further comprising
a microcontroller mounted within the head and connected to the power supply;
a memory device mounted within the head and connected to the microcontroller;
a short range transceiver mounted within the head, connected to the microcontroller, and configured to provide communication between the microcontroller and a separate computer device with a control application that provides control instructions to the microprocessor.

6. The personal hygiene device of claim 5 further comprising a loudspeaker connected to the microcontroller.

7. The personal hygiene device of claim 5 further comprising a camera mounted in the head behind a second partially mirrored area or an unmirrored area of the mirror surface connected to the microcontroller and the memory device.

8. The personal hygiene device of claim 1 further comprising
a charging port positioned in the base;
a first induction coil positioned in the base and connected to the charging port; and
an opposing second induction coil connected to the power supply and positioned in the head in a location adjacent to the first induction coil when the telescoping arm is in a collapsed position and the head is positioned adjacent to the base.

9. A personal hygiene device comprising
a head having a first side and a second side, the head comprising
a mirror positioned on the first side, and
a light positioned between the mirror and the second side;
a weighted base having a top side and a bottom side, the base comprising
a well-defined within the top side of the weighted base; and
an extendible and collapsible arm operatively connected between the head and to the base and configured to be received in the well when the arm is fully collapsed,
wherein the first side of the head contacts the top side of the base when the arm is fully collapsed.

10. The personal hygiene device of claim 9, wherein the first side has a first perimeter and the top side has a top perimeter and the first perimeter is substantially the same as the top perimeter.

11. The personal hygiene device of claim 9, wherein at least a portion of the bottom side is constructed of a slip-resistant material.

12. The personal hygiene device of claim 9, wherein the arm is rotatable at least 90 degrees with respect to a horizontal plane of the base.

13. The personal hygiene device of claim 9, wherein when the arm is at least partially extended, the head is rotatable substantially omnidirectionally with respect to the arm.

14. The personal hygiene device of claim 9, wherein the head further comprises a first magnetic component, the base further comprises a second magnetic component, and the two magnetic components are configured to be attracted to each other to permit a secure closure of the head against the base.

15. A method of using a personal hygiene device comprising a head with a mirrored surface and housing a light source therein positioned to shine through a first area of the mirrored surface, a base, and a telescoping arm connecting the head and the base, the method comprising
placing the base of the device on a surface;
lifting up on the head to dissociate the head from the base;
withdrawing the arm from the base; and
extending the arm to a desired length,
automatically actuating the light source in the head when the head is dissociated from the base.

16. The method of claim 17 further comprising rotating the arm with reference to the base, wherein the arm rotates at least 90 degrees from a horizontal plane of the base.

17. The method of claim 17 further comprising rotating the head omnidirectionally with respect to the arm.

18. The method of claim 17 further comprising touching the mirror to change a status of the light.

19. The method of claim 17, wherein
the device further comprises a camera positioned within the head behind a second area of the mirrored surface; and wherein
the method further comprises capture images incident on the mirrored surface with the camera.

20. The method of claim 17 further comprising visualizing an anatomical area of interest while performing an activity selected from dermatological examination, self-catheterization, Kegel exercises, and sexual activity.
